(19) United States
(12) Patent Application Publication
(10) Pub. No.: US 2002/0013539 A1
(43) Pub. Date: Jan. 31, 2002

(54) Methods and devices for diagnosis of precancer and cancer in breast milk ducts

(76) Inventor: David Hung, Belmont, CA (US)

Correspondence Address: BANNER & WITCOFF, 1001 G STREET N W, SUITE 1100, WASHINGTON, DC, 20001, US

(21) Appl. No.: 09/912,499

(22) Filed: Jul. 26, 2001

Related U.S. Application Data
(60) Provisional application No. 60/221,654, filed on Jul. 28, 2000.

(51) Int. Cl. A61B 5/00; B65D 81/00

(52) U.S. Cl. 600/573

(57) ABSTRACT

The invention provides devices, methods and systems for retrieving and analyzing a sample of breast duct fluid (including ductal epithelial cells and other ductal contents) for the purpose of diagnosing a breast condition, including the conditions of cancer or precancer. All or part of the analysis can be conducted in situ. The items analyzed can be quantified.

### CROSS-REFERENCES TO RELATED APPLICATIONS

This application also claims the benefit under 37 CFR 1.78 of provisional application No. 60/221,654 filed on Jul. 28, 2000. The full disclosures of the prior application are incorporated herein by reference.

### INTRODUCTION

1. Field of the Invention

The field of this invention is breast duct access to analyze a ductal fluid sample to detect breast precancer or cancer. Analysis can be performed in situ or by retrieving a sample from the breast.

2. Background

Originally, retrieval of ductal fluid was facilitated by collecting the fluid from spontaneous nipple discharge, and later from nipple aspiration. Papanicolaou et al (1958) Cancer, 11:377-409 describes exfoliative cytology from spontaneous nipple discharge of the human mammary gland and its value in the diagnosis of breast cancer. Goodson WH & King EB, Chapter 4: Discharges and Secretions of the Nipple, The Breast: Comprehensive Management of Benign and Malignant Diseases (1998)2^{nd} Ed. vol 2, Bland & Kirby Eds. W. B. Saunders Co, Philadelphia, Pa. pp. 51-74 describes nipple discharge and the ways in which it has been used to characterized conditions of the breast. Nipple aspirate cytology for the study of breast cancer precursors is described in King et al, (1983) Journal of the National Cancer Institute 71(6): 1115-21. Cytological epithelial hyperplasia and atypical hyperplasia diagnosed in nipple aspirate fluid are associated with increased risk of breast cancer in a study of 2701 women as described in Wrensch et al, (1992) Am. J. Epidemiology, v. 135 (2): 130-141. Nipple aspirate fluid is identified as a promising non-invasive method to identify cellular markers of breast cancer risk in Sauter et al, (1997) British Journal of Cancer 76(4): 494-501.

Diagnosis using ductal fluid retrieved by accessing the duct with a lumen-based device such as a catheter or cannula is described by Sartorius et al (1977) which proposed cytologic evaluation of breast fluid retrieved using hair-like single-lumen catheters for the detection of breast disease as described in Journal of the National Cancer Institute 59(4): 1073-80. Love and Barsky, (1996) Lancet 348(9033): 997-9 demonstrated retrieval of ductal fluid by breast-duct endoscopy using a single-lumen device to study stages of cancerous breast disease. A Company called Diagnostics, Inc. formed in 1968, produced devices to obtain breast ductal fluid for cytological evaluation. The devices included a nipple aspiration device to collect NAF from subjects, and single-lumen catheters to retrieve ductal fluid. The devices were sold prior to May 28, 1976 for the purpose of collecting breast ductal fluid for cytological evaluation.

By the procedure of ductal lavage, ductal epithelial cells that line the walls of the ductal lumen are washed out of the duct. Lavage or wash fluid is infused into the duct, and the lavage fluid mixed with ductal fluid is collected. Lavage is described in copending and co-owned applications including Ser. Nos. 09/067,661, 09/301,058, PCT US99/09141, No. 60/122,076, Ser. No. 09/313,463, No. 60/143,359, and U.S. Ser. No. 09/473,510, all incorporated by reference in their entirety. In some cases suction can be applied to the tool accessing the ductal lumen in order to retrieve a maximum amount of cells and/or fluid. Lavage or wash fluid can be infused into the duct, and collected. Suction can be applied to the tool accessing the ductal lumen in order to retrieve a maximum amount of cells and/or fluid.

Access of a breast duct can be facilitated as described in e.g. Love & Barsky, (1996) Lancet 348: 997-999, Makita et al (1991) Breast Cancer Res Treat 18: 179-188, or Okazaki et al (1991) Jpn J. Clin. Oncol. 21:188-193. Alternatively, ductal fluid can be retrieved by a medical tool, e.g. a catheter or a cannula placed into the duct to infuse wash fluid to retrieve a mixture of wash and ductal fluids. The fluid from the breast duct can contain ductal epithelial cells, including cells of a stage considered to be precancerous or cancerous.

Nipple aspiration of breast ductal fluid is achieved by using vacuum pressure. Nipple aspiration techniques are also described and claimed in co-pending and co-owned patent application U.S. Ser. No. 09/438,219, herein incorporated by reference in their entirety. Nipple aspirate fluid can be retrieved as described in e.g. Goodson W H & King E B, Chapter 4: Discharges and Secretions of the Nipple, The Breast: Comprehensive Management of Benign and Malignant Diseases (1998)2^{nd} Ed. vol 2, Bland & Kirby eds. W. B. Saunders Co, Philadelphia, Pa. pp. 51-74; Wrensch et al., (1992) American Journal of Epidemiology. 135(2):130-41; and Sauter et al (1997) British Journal of Cancer. 76(4):494-501. Ductal lavage is described in copending patent application U.S. Ser. No. 09/067,661 filed Apr. 28, 1998. Cells of the lesion can be retrieved by collecting the ductal fluid that contains some of these cells, e.g. by aspirating the nipple to obtain nipple aspirate fluid, e.g. as described in Petrakis (1993) Cancer Epidem. Biomarker Prev. 2:3-10, Petrakis (1986) Breast Cancer Res. Treat 8: 7-19, Wrensch et al (1992) Am. J. Epidem. 135:130-141, Wrensch et al (1990) Breast Cancer Res Treat 15: 39-21, and Wrensch et al (1989) Cancer Res. 49: 2168-2174. Also fluid secretions from the nipple can be collected as they spontaneously appear on the nipple surface. In order to collect the fluid not mixed with ductal fluid from other ducts, a practitioner carefully watches for the signs of fluid and retrieves the fluid from the nipple surface near the orifice before it has a chance to mix with fluid from any other orifice.

While aspiration, and cannulation or catheterization are suitable means for diagnostic retrieval of breast duct fluid, they require suction and fluid infusion respectfully. It would be a great advantage to improve upon the presently employed intraductal diagnostic devices and methods of breast cancer detection. The present invention provides this advantage.

### SUMMARY OF THE INVENTION

The invention provides a device for collection of breast duct fluid. Accordingly, there is provided a device for collection of breast duct fluid and detection of breast cancer or precancer comprising:

a probe of a diameter sufficiently small to penetrate a breast duct having a distal portion capable of contacting an interior lumen of a breast duct, wherein said distal portion can contact and retrieve a sufficient sample of the breast duct fluid for analysis, said probe unattached to a fluid source or lumen.

The distal portion of the device can comprise an absorbent material that can absorb breast duct fluid. The distal portion can comprise a collection portion that can collect the breast duct fluid it contacts. The collection portion can be tubular. The collection portion can extend some of the distance of the probe. The distal portion can comprise a surface having molecules affixed that bind an agent in the ductal fluid it contacts. The distal portion can comprise a means to measure a quality of the ductal fluid in situ. The quality can comprise an indicia selected from the group consisting of cell size, cell density, nuclear size, nucleoli size, and chromatin coarseness. The distal portion can comprise a MEMS capable of detecting in situ a quality of the ductal fluid. The quality can comprise a marker.

The device can comprise a coating of an anesthetic on the exterior of the probe.

The probe of the device can be rigid before entry into the breast duct, and flexible upon residence in the duct. The probe can comprise a shape memory material.

The invention also provides a method of using the device. Accordingly, there is provided a method of collection and analysis of breast duct fluid and detection of breast cancer or precancer comprising:

inserting a probe comprising a distal portion that can attract or collect breast duct fluid and contents; and

collecting a sample of ductal fluid into the distal portion.

The method can further comprise analyzing the sample of ductal fluid collected by the distal portion of the probe. The method can further comprise removing the probe from the breast duct and analyzing the sample of ductal fluid collected or attracted by the distal portion. Analyzing in the method can comprise contacting the distal portion comprising ductal fluid with a reagent. Analyzing can comprise cytological analysis of ductal epithelial cells. Analyzing can comprise detection of a marker. Analyzing can comprise measuring a quality of the ductal fluid or ductal cells in situ. In the method, collecting can comprise a waiting period with the probe in the duct for a period of time in a range from about a few seconds to a few weeks.

The invention also provides a system of collection and analysis of breast duct fluid and detection of breast cancer or precancer comprising:

a device comprising a probe for accessing a breast duct having a distal portion for collecting or attracting ductal fluid and/or ductal cells;

reagents for contacting the distal portion for detection of a marker or analysis of the ductal fluid sample, and

instructions for use of the system to diagnose breast cancer or precancer in a breast duct.

The invention also provides an article for collection of breast duct fluid and detection of breast cancer or precancer comprising a receiving unit of a sufficient dimension to isolate a breast duct opening on a nipple surface, wherein said unit can contact a bead of ductal fluid on the nipple surface at the ductal orifice after nipple aspiration of said nipple. The unit can absorb the aspirated ductal fluid from the nipple surface for analysis.

The invention also provides a method of collection and analysis of breast duct fluid and detection of breast cancer or precancer comprising contacting a ductal orifice having a bead of ductal fluid on a nipple surface with a receiving unit of a sufficient dimension to isolate the ductal orifice, whereupon said unit absorbs the ductal fluid for analysis.

### BRIEF DESCRIPTION OF THE FIGURES

FIG. 1 shows a cross section of two breast ducts in a breast.

FIG. 2 shows a probe having a distal portion accessing a breast duct to the lactiferous sinus.

FIG. 3 shows a probe made of shape memory material having a distal portion accessing distal to the lactiferous sinus, and yielding to the ductal architecture during residence therein.

FIG. 4 shows a receiving unit for collecting a bead of ductal fluid at a ductal orifice on a nipple surface.

### DETAILED DESCRIPTION OF PREFERRED EMBODIMENTS OF THE INVENTION

The following preferred embodiments and examples are offered by way of illustration and not by way of limitation.

Breast cancer proceeds through discrete premalignant and malignant cellular stages: normal ductal epithelium, atypical ductal hyperplasia, ductal carcinoma in situ (DCIS) (including low grade DCIS and high grade DCIS), and finally invasive ductal carcinoma. The line between cancer and precancer is defined differently in the field, but precancer can include those conditions up through low grade DCIS, and cancer can include high grade DCIS and invasive carcinoma. The condition can arise in the breast duct lumen or in the terminal ductal lobular unit (TDLU).

The device for collection of breast duct fluid and detection of breast cancer or precancer can comprise a probe. The probe can have a diameter sufficiently small to penetrate a breast duct. Thus, the size of the probe lumen or shaft can be in a range from about 0.008 cm to about 0.040 cm in diameter; and preferably the probe diameter is in a range from about 0.012 cm to about 0.025 cm. The probe can have a graduated diameter, e.g. a smaller diameter distally expanding to a greater diameter proximally. The probe can be made of metal, plastic or polymer or other suitable material for sufficient rigidity for ductal access, and also sufficiently adaptable to retrieving a ductal fluid sample at the distal end, and if need be sufficiently flexible to be retained in the breast duct.

The distal portion of the probe, the portion that initially accesses the breast duct and penetrates to a sufficient depth to contact or retrieve a sample of ductal fluid is capable of contacting ductal fluid or ductal contents within an interior lumen of a breast duct. Thus, its diameter might be smaller than the rest of the probe member, or it may be the same size. It may also be expandable to a larger size or diameter within the duct. The distal portion can contact and retrieve a sufficient sample of the breast duct fluid for analysis, e.g. by absorbing some fluid, contacting the fluid and allowing specific binding of marker molecules to a binding partner affixed to the distal portion of the probe, by promoting the uptake of a small amount of the ductal fluid into a lumen or cavity in the distal portion of the probe, such quantity sufficient for in situ or later analysis of the contents of the captured fluid. The probe member is and remains unattached to a fluid source or external lumen that might attempt to draw up the ductal fluid or infuse wash fluid. Thus the probe acts like a “dipstick” to test the contents of the ductal passage in a breast duct, by retrieving a small sample of the ductal fluid and providing a means to analyze it either in situ in the duct upon contact with reagent in the distal tip, or upon withdrawal of the probe from the duct and contact with detecting or other reagent thereafter.

The distal portion of the probe can comprise an absorbent material that can absorb breast duct fluid. For example, the material can comprise cotton, or other spun materials, or other fluid absorbing material such as an absorbent hydrogel, absorbent nylon, or the like.

The distal portion can comprise a collection portion that can collect the breast duct fluid it contacts. The collection portion can be a lumen that pulls a small volume of ductal fluid into it by physical action, e.g. capillary action, pulling the fluid into a small lumen in the distal end of the probe. Accordingly the collection portion can be tubular. The collection portion can be a bulb, space of other shape, or other cavity at the distal end of the probe. It may be a fixed size or expandable upon contact with fluid, or upon filling with fluid. The collection portion may extend some of the distance of the probe, thus beginning at the distal tip and extending back up the probe toward the ductal orifice (when the probe is in the duct).

The distal portion of the probe can comprise a surface having molecules affixed to it that bind an agent in the ductal fluid that is contacted in the duct. For example, the probe can be coated with an antibody specific for an antigen in ductal fluid (e.g. an antigen present on the surface of ductal epithelial cells; a soluble antigen secreted into the ductal lumen from neoplastic cells present in a lesion in the duct, etc.). By placing the probe coated with antibody in the ductal lumen, the antibodies contact ductal fluid having the antigen sought and the antigen binds the antibody. Upon removal of the probe, the antigen binding is detected and evaluated for what that binding indicates about the condition of the duct. For example, while the probe is in the duct and after it contacts ductal fluid an agent in the ductal fluid may contact a reagent on or in the probe and react by binding or otherwise reacting with the detecting reagent. For example, a change in color may be noted on a strip.

The method of collection and analysis of breast duct fluid and detection of breast cancer or precancer can comprise inserting a probe comprising a distal portion that can attract breast duct fluid and contents. The draw tip of the distal portion of the probe of the device can comprise an attractor, panning or magnetism, such as for example driven processes, including but not limited to heat, magnetics, other attraction forces, or other active attraction mechanism. Attracted molecules, cells, agents or fluid can be trapped on the device after being attracted to it. Attracted molecules, cells, agents or fluid can be analyzed on the probe in situ (e.g. by color change, or other identifying mechanism facilitated on the probe itself), of may be removed in or on the device after collection for completing an analysis outside the breast.

In all cases of collection and/or attraction of molecules, cells, agents or fluid, quantification is possible by standard biochemical quantification methods that vary depending on the entity being quantified. The distal portion can comprise a means to measure a quality of the ductal fluid in situ. The quality to be detected can comprise an indicia such as for example cell size, cell density, nuclear size, nucleoli size, and chromatin coarseness. The quality can also be a marker. In order to detect a quality of the ductal fluid in situ, the distal portion can comprise a microelectromechanical systems (MEMS) capable of detecting in situ a quality of the ductal fluid. MEMS are described for example in Mechanical Engineering, 118 (no. 10):65-68 (1996).

The probe can also comprise a coating of an anesthetic on the exterior of the probe. The coating provides anesthetic action at the contact points in the ductal orifice and ductal lumen to provide anesthesia in the duct during the access and retrieval of a ductal fluid sample.

The probe can be made of a material that provides that it is rigid before entry into the breast duct, and that it then becomes flexible upon residence in the duct. Thus, the probe can be made of a thermo-sensitive polymer that is stiff at room temperature, and which softens and become flexible at body temperature. This feature provides a probe that can reside in a duct over a period of time exceeding an office visit, e.g. for several hours, or several days, or several weeks. The residence feature allows for the collection and sampling of a sufficient quantity of ductal fluid for making a particular analysis. An example of material that the probe can comprise to achieve this feature is a shape memory material, such as, for example a nickel titanium alloy material.

The method of collection and analysis of breast duct fluid and detection of breast cancer or precancer comprises inserting a probe comprising a distal portion that can attract or collect breast duct fluid and contents; and collecting a sample of ductal fluid into the distal portion. The probe is not connected to a lumen to infuse or retrieve fluid externally (outside the probe) into another receptacle. The sample collected by the distal portion of the probe can be analyzed, such as described herein, e.g. for markers, or for other indicia of changed quality in the ductal fluid. For example, indicia such a change in cell size, cell density, nuclear size, nucleoli size, and chromatin coarseness can be detected in the fluid retrieved or contacted by the probe.

The probe can be removed from the breast duct and the sample collected by or attracted into the probe device can be analyzed. The analysis can comprise detection of a marker and/or detection of other indicia as describe herein. Analyzing can comprise contacting the distal portion of the probe that retains the ductal fluid with a reagent. The reagent can be, for example, an antibody, a dye, a receptor, a binding agent, a label, or other reagent capable of facilitating detection of the marker or indicia sought. Analyzing can comprise cytological analysis of ductal epithelial cells retrieved. Analyzing can comprise measuring a quality of the ductal fluid or ductal cells in situ, such as measuring a cell size, cell density, nuclear size, nucleoli size, and/or chromatin coarseness.

Collecting can comprise introducing a waiting period by keeping the probe in the duct for a period of time in a range from about a few seconds to a few weeks. The waiting period provides the opportunity for the probe to retrieve, absorb, collect, and/or attract ductal contents. The ductal contents can include, as describe elsewhere, ductal fluid, markers, molecules, cells and/or indicia or qualities (such as size and shape) inherent in these contents.

The invention also provides a system of collection and analysis of breast duct fluid and detection of breast cancer or precancer comprising a device comprising a probe for accessing a breast duct having a distal portion for collecting or attracting ductal fluid and/or ductal cells. A system will also comprise reagents for contacting the distal portion for detection of a marker or analysis of the ductal fluid sample, and instructions for use of the system to diagnose breast cancer or precancer in a breast duct. The reagents can be, e.g. test or analysis components such as antibodies, binding agents, receptors, labels, dye, washing agents and other materials that may be necessary in order to detect a marker or indicia of breast precancer or cancer.

In addition to probing and collecting from inside the ductal lumen, the invention provides an article for collection of breast duct fluid on the surface of the nipple. Collected ductal fluid can be analyzed for detection of breast cancer or breast precancer. The article comprises a receiving unit of a sufficient dimension to isolate a breast duct opening on a nipple surface. The receiving unit may be absorbent, i.e. an absorbent pad. The receiving unit can be a flattish surface such as a flattish pad or it can be a probe-like article having a rounded tip for contacting a bead of ductal fluid at a ductal orifice on a nipple surface. After aspiration of the nipple, fluid is yielded from one or more ducts on the nipple surface. The receiving unit can contact a bead of ductal fluid on the nipple surface at the ductal orifice after nipple aspiration of the nipple. The receiving unit can absorb the aspirated ductal fluid from the nipple surface for analysis of breast cancer or precancer markers. Each duct that yields fluid can be contacted with its own receiving unit, and thus the different fluid specimens from the different ducts can be kept separate.

In accordance with the article for collecting ductal fluid on the nipple surface, the invention also provides a method of collection and analysis of breast duct fluid and detection of breast cancer or precancer comprising contacting a ductal orifice on a nipple surface. After aspiration of the nipple, one or more ductal orifices can have a bead of ductal fluid at the orifice on a nipple surface. The bead can be contacted with a receiving unit of a sufficient dimension to isolate the ductal orifice. The receiving unit can be a pad or an absorbent probe tip, as described above. The receiving unit then can absorb or otherwise collect the ductal fluid for analysis from the ductal orifice from which it was produced during nipple aspiration, and the collected ductal fluid can be analyzed for markers as described herein.

The agent or marker sought in the ductal fluid can be, e.g. a molecule such as an antibody, a peptide, a polypeptide, a nucleic acid, a polynucleotide, a small organic molecule, a macromolecule, a polymer, a carbohydrate, or a lipid. In general any marker characteristic of ductal precancer or cancer can be used, provided it is retrievable by the probe. The cells retrieved can also be analyzed by cytological analysis. Other cytological criteria and processes related to ductal fluid analysis are described in Barret et al, Acta Cytol 1976;20:174-180; Goodson et al, Discharges and Secretions of the Nipple, THE BREAST: COMPREHENSIVE MANAGEMENT OF BENIGN AND MALIGNANT DISEASES, Second Edition, Vol. 1, Chapter 4, page 1; King et al, Cytometry 1984; 5: 124-130; King et al, A.J.C.P. 1975; 64: 739-748; King et al, A.J.C.P. 1975; 64: 728-738; King et al, Cytopathology of Abnormal Mammary Duct Epithelium, Prevention and Detection of Cancer, Part II, Detection, vol 2 Cancer detection in specific sites, 1976; King et al, J Natl Cancer Inst, 1983; 71: 1115-1121; Kjellgren et al, Acta Cytol 1964; 8: 216-217; Masood et al, The Breast Journal 1999; 5:1-2; Papanicolaou et al, Cancer 1958; 377-409; Petrakis et al, Cancer Epidemiology, Biomarkers and Prevention 1993; 2:3-10; Ringrose et al, Acta Cytol 1966; 10:373-375; Sartorius et al, NC11977; 59:1073-1080; Sauter et al, British J. Cancer 1997; 76(4):494-501; Wrensch et al, Amer J. Epid. 1992; 135: 130-141.

Once the ductal fluid is analyzed for one or more markers, the fluid may also be analyzed cytologically to determine the cytological status of the ductal epithelial cells and other cells. Cytological assays that can be performed on the cells retrieved from a duct or from nipple aspirate can include e.g. assays described in King et al, J. Nat'l Cancer Inst (1983) 71:1115-21, Wrensch etal. (1992) Am. J. Epidem. 135: 130-141, Papanicolaou et al, (1958) Cancer, 11:377-409 and Goodson W H & King E B, Chapter 4: Discharges and Secretions of the Nipple, THE BREAST: COMPREHENSIVE MANAGEMENT OF BENIGN AND MALIGNANT DISEASES (1998)2^{nd} Ed. vol 2, Bland & Kirby eds. W. B. Saunders Co, Philadelphia, Pa. pp. 51-74. For example, as described in Goodson and King (page 60) atypical hyperplasia presents as having cellular abnormalities, increased coarseness of the chromatin, and tendency for more single cells as well as groups of cells. With regard to carcinoma in situ, Papanicolaou et al, described cellular abnormalities, e.g. nuclear abnormalities diagnosed by cytology of fluid from nipple secretions containing ductal cells. The cytology of abnormal cells can also be conducted as described in Sartorius et al (1977) J. Natl Cancer Inst 59: 1073-1080. and King et al, (1983) JNCI71(6) 1115-1121. Atypia and carcinoma in situ are widely characterized pathologically, as described in Page et al, (1998) Mod Pathol 11(2): 120-8. The ductal fluid can be analyzed by cytological techniques by placing some of the fluid on a slide with a standard cytological stain using a light microscope. The cells can be studied for atypical growth patterns in individual cells and clusters of cells using published methods, including Mouriquand J, (1993) S Karger Pub, “Diagnosis of Non-Palpable Breast Lesions: Ultrasonographically Controlled Fine-Needle Aspiration: Diagnostic and Prognostic Implications of Cytology” (ISBN 3805557477); Kline T S and I K, Pub Igaku-Shoin Medical “Breast: Guides to Clinical Aspiration Biopsy” (LSBN 0896401596; Masood, American Society of Clinical Pathology: November 199S, “Cytopathology of the Breast” ISBN 0891893806; and Feldman P S, American Society of Clinical Pathology, November 1984, “Fine Needle Aspiration Cytology and Its Clinical Applications: Breast and Lung” ISBN 0891891846.

Other references that discuss cytological analysis and which give guidance to an analysis of ductal epithelial cells derived from ductal fluid include Silverman et al, (Can FNA biopsy separate atypical hyperplasia, carcinoma in situ, and invasive carcinoma of the breast?: Cytomorphologic criteria and limitations in diagnosis, Diagnostic Cytopathology) 9(6):713-28, 1993; Masood et al, (Immunohistochemical differentiation of atypical hyperplasia vs. carcinoma in situ of the breast) Cancer Detection & Prevention. 16(4):225-35, 1992; Masood et al, (Cytologic differentiation between proliferative and nonproliferative breast disease in mammographically guided fine-needle aspirates) Diagnostic Cytopathology. 7(6):581-90, 1991; Masood S., (Occult breast lesions and aspiration biopsy: a new challenge) Diagnostic Cytopathology. 9(6):613-4, 1993; Masood S., (Prognostic factors in breast cancer: use of cytologic preparations) Diagnostic Cytopathology. 13(5):388-95, 1995; Novak and Masood, (Nuclear grooves in fine-needle aspiration biopsies of breast lesions: do they have any significance?) Diagnostic Cytopathology. 18(5):333-7, 1998; Sidawy et al, (Interobserver variability in the classification of proliferative breast lesions by fine-needle aspiration: results of the Papanicolaou Society of Cytopathology Study) Diagnostic Cytopathology. 18(2):150-65, 1998; Masood et al, (Automation in cytology: a survey conducted by the New Technology Task Force, Papanicolaou Society of Cytopathology) Diagnostic Cytopathology. 18(1):47-55, 1998; and Frykberg and Masood Copeland E M 3d. Bland K I., (Ductal carcinoma in situ of the breast) Surgery, Gynecology & Obstetrics 177(4):425-40, 1993.

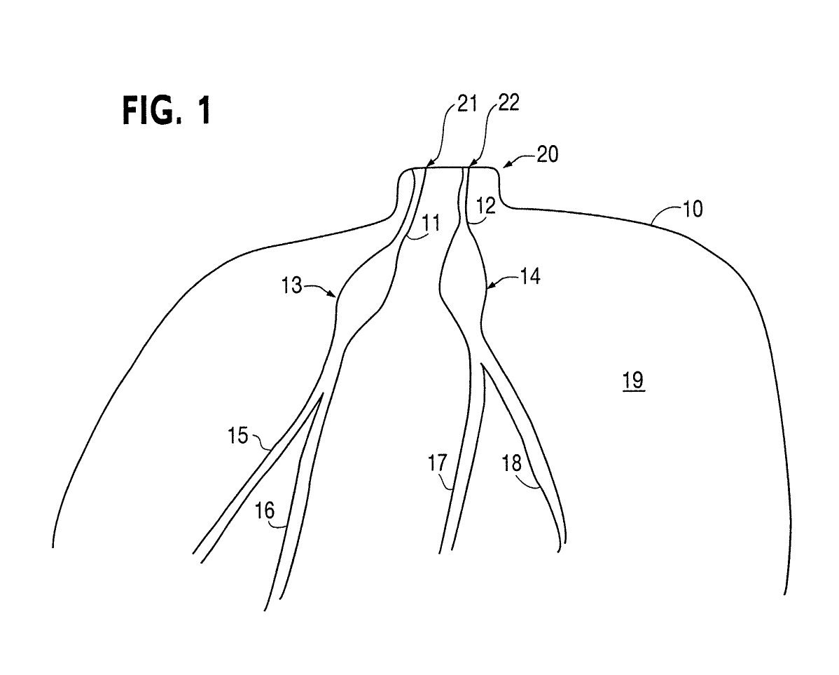

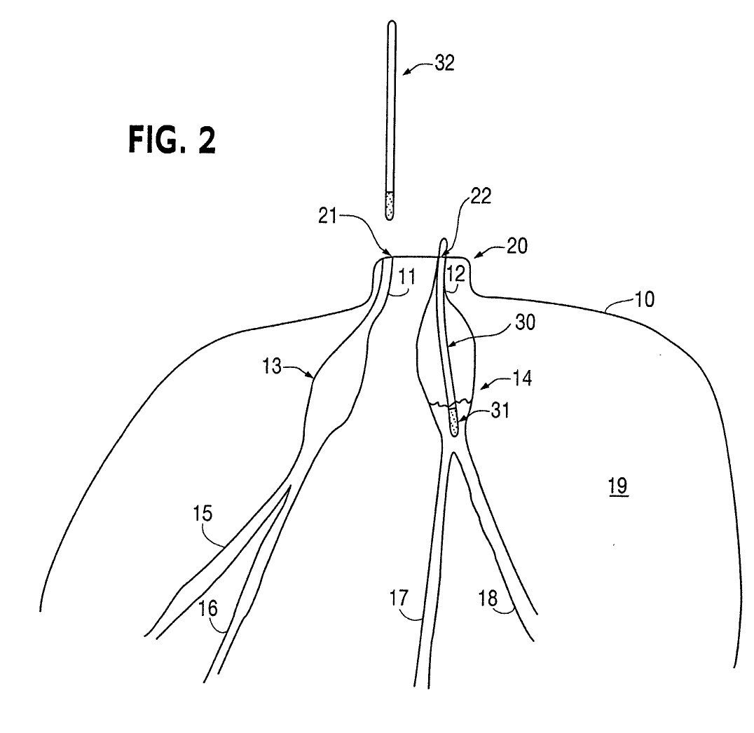

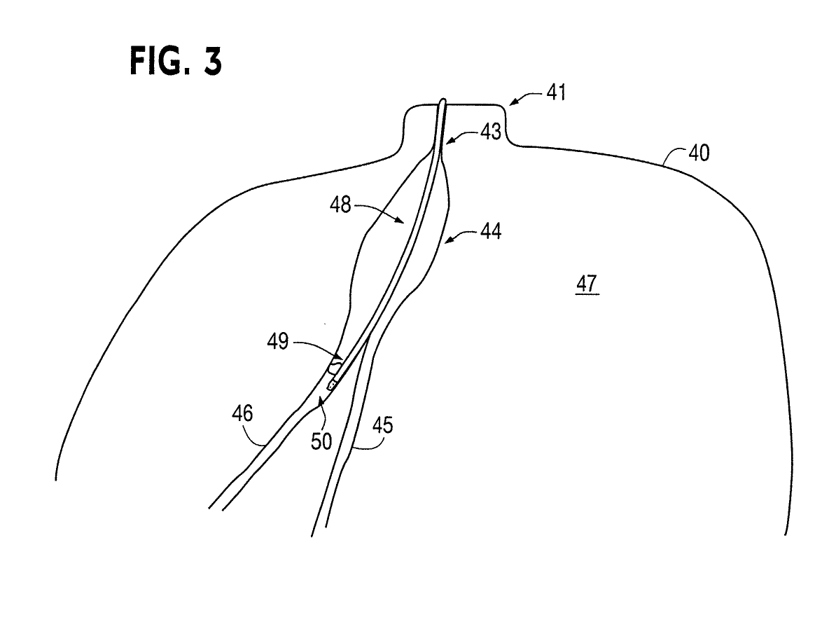

Turning now to the Figures. FIG. 1 illustrates a cross section of a breast 10, indicating two breast ducts, 11 and 12, having lactiferous sinus 13 and 14, and branching lumens 15, 16, 17, and 18. Breast tissue surrounds the ducts 19. The ducts are accessed by nipple surface 20 through ductal orifices 21 and/or 22. FIG. 2 demonstrates the same breast having two ducts shown in cross section. Duct 12 is accessed with device 30 having distal end 31 contacting ductal fluid residing in the lactiferous sinus. Duct 11 is targeted for access by device 32 having distal end 33. Ductal fluid and contents are absorbed into the distal end 31 accessing lactiferous sinus 14. FIG. 3 demonstrates breast 40 accessed at nipple surface 41, through ductal orifice 42 into duct 43 and lactiferous sinus 44, and ductal branch 46 by device 48 having distal tip 49 which contacts ductal fluid and ductal contents 50 while bending to the contours of the ductal passage using the shape memory properties of the probe material. Eventually probe 48 is withdrawn retrieving ductal contents 50 that contacted and absorbed into tip 49 in ductal passage 46.

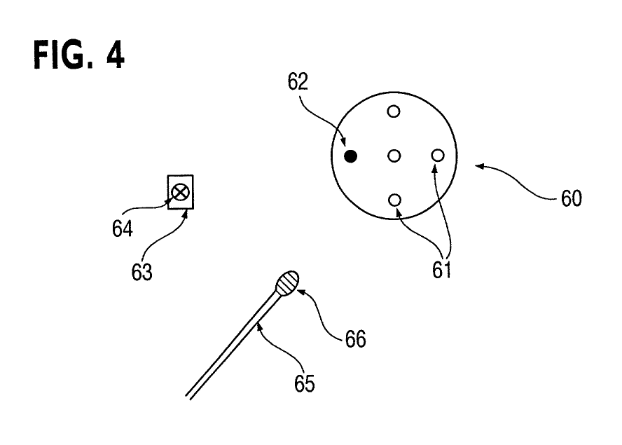

FIG. 4 depicts a nipple surface 60 having non-targeted ducts 61 and a target duct 62. Target duct 62 comprises a bead of ductal fluid generated by aspiration of the nipple surface. Article 63 is a small unit comprising a pad having absorbent region 64 for contacting the bead at the ductal orifice on the nipple surface. Article 65 is a probe-like or stick design having an absorbent tip 66 for contacting with the bead of ductal fluid at the ductal orifice on the nipple surface.

### EXAMPLES

1. Breast Duct Fluid Sampling Using “Dipstick” Device

The right and left breasts of a woman at high risk for breast cancer (e.g. age 35; family history of breast cancer) are prepared with alcohol and dekeratinized with a dekeratinizing agent. Two breast ducts on each breast are identified. The ductal orifice is numbed on contact with drops of concentrated lidocaine. A “dipstick” device coated on the exterior with a viscous high concentration lidocaine formulation and having a distal end for accessing a breast duct and contacting and absorbing ductal fluid is placed at the ductal orifice, and pushed into the duct. The probe device is gently moved deeper into the duct and allowed to remain in the duct, preferably distal to the sphincter of the lactiferous sinus for sufficient time for the distal tip to collect ductal fluid and contents (e.g. about 10 minutes). The other target ducts are similarly accessed. The probes are removed and the tips of the probes placed in reagent to extract and preserve the ductal contents. The cells are isolated and placed on a cytological slide for analysis. The remaining eluant is treated for identification of several soluble markers in binding assays to known antibodies which recognize the marker.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A device for collection of breast duct fluid and detection of breast cancer or precancer comprising:
a probe of a diameter sufficiently small to penetrate a breast duct having a distal portion capable of contacting an interior lumen of a breast duct, wherein said distal portion can contact and retrieve a sufficient sample of the breast duct fluid for analysis, said probe unattached to a fluid source or lumen.

2. A device as in claim 1, wherein the distal portion comprises an absorbent material that can absorb breast duct fluid.

3. A device as in claim 1, wherein the distal portion comprises a collection portion that can collect the breast duct fluid it contacts.

4. A device as in claim 3, wherein the collection portion is tubular.

5. A device as in claim 3, wherein the collection portion extends some of the distance of the probe.

6. A device as in claim 1, wherein the distal portion comprises a surface having molecules affixed that bind an agent in the ductal fluid it contacts.

7. A device as in claim 1, wherein the distal portion comprises a means to measure a quality of the ductal fluid in situ.

8. A device as in claim 7, wherein the quality comprises an indicia selected from the group consisting of cell size, cell density, nuclear size, nucleoli size, and chromatin coarseness.

9. A device as in claim 1, wherein the distal portion comprises a MEMS capable of detecting in situ a quality of the ductal fluid.

10. A device as in claim 9, wherein the quality comprises a marker.

11. A device as in claim 1, further comprising a coating of an anesthetic on the exterior of the probe.

12. A device as in claim 1, wherein the probe is rigid before entry into the breast duct, and flexible upon residence in the duct.

13. A device as in claim 1, wherein the probe comprises a shape memory material.

14. A method of collection and analysis of breast duct fluid and detection of breast cancer or precancer comprising:
inserting a probe comprising a distal portion that can attract or collect breast duct fluid and contents; and
collecting a sample of ductal fluid into the distal portion.

15. A method as in claim 14, further comprising analyzing the sample of ductal fluid collected by the distal portion of the probe.

16. A method as in claim 14, further comprising removing the probe from the breast duct and analyzing the sample of ductal fluid collected or attracted by the distal portion.

17. A method as in claim 14, wherein analyzing comprises contacting the distal portion comprising ductal fluid with a reagent.

18. A method as in claim 14, wherein analyzing comprises cytological analysis of ductal epithelial cells.

19. A method as in claim 14, wherein analyzing comprises detection of a marker.

20. A method as in claim 14, wherein analyzing comprises measuring a quality of the ductal fluid or ductal cells in situ.

21. A method as in claim 14, wherein collecting comprises a waiting period with the probe in the duct for a period of time in a range from about a few seconds to a few weeks.

22. A system of collection and analysis of breast duct fluid and detection of breast cancer or precancer comprising:
a device comprising a probe for accessing a breast duct having a distal portion for collecting or attracting ductal fluid and/or ductal cells;
reagents for contacting the distal portion for detection of a marker or analysis of the ductal fluid sample, and
instructions for use of the system to diagnose breast cancer or precancer in a breast duct.

23. An article for collection of breast duct fluid and detection of breast cancer or precancer comprising:
a receiving unit of a sufficient dimension to isolate a breast duct opening on a nipple surface, wherein said unit can contact a bead of ductal fluid on the nipple surface at the ductal orifice after nipple aspiration of said nipple.

24. The article as in claim 23, wherein the unit can absorb the aspirated ductal fluid from the nipple surface for analysis.

25. A method of collection and analysis of breast duct fluid and detection of breast cancer or precancer comprising:
contracting a ductal orifice having a bead of ductal fluid on a nipple surface with a receiving unit of a sufficient dimension to isolate the ductal orifice, whereupon said unit absorbs the ductal fluid for analysis.
